(19) United States
(12) Patent Application Publication
(10) Pub. No.: US 2025/0104846 A1
(43) Pub. Date: Mar. 27, 2025

(54) INFORMATION PROCESSING APPARATUS, INFORMATION PROCESSING METHOD, AND PROGRAM

(71) Applicant: FUJIFILM CORPORATION, Tokyo (JP)

(72) Inventors: Hisatsugu HORIUCHI, Tokyo (JP); Takeru SUGIYAMA, Tokyo (JP); Ryoji SASADA, Tokyo (JP); Takeyasu KOBAYASHI, Tokyo (JP); Tomohiro NOMURA, Tokyo (JP)

(21) Appl. No.: 18/815,767

(22) Filed: Aug. 26, 2024

(30) Foreign Application Priority Data
Sep. 25, 2023 (JP) 2023-161763

(51) Int. Cl. G16H 30/40; G10L 25/57; G11B 27/34

(52) U.S. Cl. CPC G16H 30/40; G10L 25/57; G11B 27/34

(57) ABSTRACT

The information processing apparatus includes at least one processor. The processor acquires a series of radiation images captured by performing continuous irradiation with radiation, generates, in a case in which a voice uttered during an imaging period of the series of radiation images is a specific voice, image specification information for specifying a radiation image selected from among the series of radiation images according to a timing of occurrence of the voice, and associates the image specification information with the series of radiation images.

### CROSS-REFERENCE TO RELATED APPLICATIONS

The present application claims priority under 35 U.S.C. § 119 to Japanese Patent Application No. 2023-161763, filed on Sep. 25, 2023. The above application is hereby expressly incorporated by reference, in its entirety, into the present application.

### BACKGROUND

### 1. Technical Field

The disclosed technology relates to an information processing apparatus, an information processing method, and a program.

### 2. Description of the Related Art

The following technologies are known as technologies relating to image processing using a voice. For example, JP2012-217632A discloses an image processing apparatus comprising an imaging unit that images an examination target, an image processing unit that creates a video signal from an image captured by the imaging unit, a recording unit that creates and records a video file using the video signal and a voice signal related to movements and operations of an operator who operates the imaging unit and the examination target during imaging, a voice analysis unit that monitors whether or not a voice signal intensity is equal to or greater than a predetermined threshold value, and a notification unit that notifies the operator that the voice signal intensity is equal to or greater than the predetermined threshold value.

JP2010-253017A discloses a method of storing in advance intravascular ultrasound video images, angiography video images, voice data including names of parts of imaging positions shown in frames of the intravascular ultrasound video images, and anatomical data indicating traveling patterns of blood vessels and information indicating each part of the blood vessels, detecting a part that is called out from the voice data, detecting a frame of an intravascular ultrasound video image that was captured when that part was called out, and associating the detected part with the frame.

### SUMMARY

A videofluoroscopic examination of swallowing is known as one type of examination using radiation images. A videofluoroscopic examination of swallowing is an examination to evaluate a process and a state when an examinee swallows a sample. In a videofluoroscopic examination of swallowing, a video composed of a series of radiation images captured by continuously irradiating an examinee with radiation is used.

The videofluoroscopic examination of swallowing includes the following processes.
(1) Positioning of examinee
(2) Provision of sample to examinee
(3) Chewing of sample
(4) Start of swallowing
(5) End of swallowing

In a videofluoroscopic examination of swallowing, radiation images captured in a period of interest required for diagnosis (for example, the period from (4) the start of swallowing to (5) the end of swallowing) are the main target of observation. However, the series of radiation images captured in a videofluoroscopic examination of swallowing may also include images captured during periods other than the period of interest (for example, the period from (1) positioning the examinee to (3) chewing of the sample). In this case, in order to observe images during the period of interest, it is necessary to search for the images during the period of interest from among a series of radiation images, which places a heavy burden on the observer.

The disclosed technology has been made in consideration of the above points, and an object thereof is to facilitate access to a desired image from a series of radiation images.

According to an aspect of the disclosed technology, there is provided an information processing apparatus comprising at least one processor. The processor is configured to: acquire a series of radiation images captured by performing continuous irradiation with radiation; generate, in a case in which a voice uttered during an imaging period of the series of radiation images is a specific voice, image specification information for specifying a radiation image selected from among the series of radiation images according to a timing of occurrence of the voice; and associate the image specification information with the series of radiation images.

The specific voice may be an uttered voice including a specific word. The specific voice may be a specific biological sound.

The processor may be configured to: generate a plurality of pieces of the image specification information based on a plurality of voices uttered intermittently during the imaging period of the series of radiation images; and associate the plurality of pieces of image specification information with the series of radiation images.

The processor may be configured to display a time position of the radiation image specified by the image specification information in a video composed of the series of radiation images. The processor may be configured to change the image specification information based on a change operation of a time position of the radiation image specified by the image specification information in a video composed of the series of radiation images.

The processor may be configured to: acquire a series of optical images that are continuously captured in parallel with the capturing of the series of radiation images; and play back each of a radiation video composed of the series of radiation images and an optical video composed of the series of optical images such that images at the same time point are displayed.

The processor may be configured to: extract some radiation images from the series of radiation images with the radiation image specified by the image specification information as a starting point or an ending point; and generate a data file including the extracted some radiation images. The processor may be configured to: acquire relevant information related to imaging content of the series of radiation images; and generate a data file in which the relevant information and the some radiation images are associated with each other.

According to another aspect of the disclosed technology, there is provided an information processing method executed by at least one processor included in an information processing apparatus, the information processing method comprising: acquiring a series of radiation images captured by performing continuous irradiation with radiation; generating, in a case in which a voice uttered during an imaging period of the series of radiation images is a specific voice, image specification information for specifying a radiation image selected from among the series of radiation images according to a timing of occurrence of the voice; and associating the image specification information with the series of radiation images.

According to still another aspect of the disclosed technology, there is provided a program for causing at least one processor included in an information processing apparatus to execute: acquiring a series of radiation images captured by performing continuous irradiation with radiation; generating, in a case in which a voice uttered during an imaging period of the series of radiation images is a specific voice, image specification information for specifying a radiation image selected from among the series of radiation images according to a timing of occurrence of the voice; and associating the image specification information with the series of radiation images.

According to the disclosed technology, it is possible to easily access a desired image from a series of radiation images.

### BRIEF DESCRIPTION OF THE DRAWINGS

Exemplary embodiments according to the technique of the present disclosure will be described in detail based on the following figures, wherein:

FIG. 1 is a diagram showing an example of a configuration of a radiography system according to an embodiment of the disclosed technology;

FIG. 2 is a diagram showing an example of a configuration of a radiography apparatus according to the embodiment of the disclosed technology;

FIG. 3 is a diagram showing an example of a hardware configuration of an information processing apparatus according to the embodiment of the disclosed technology;

FIG. 4 is a functional block diagram showing an example of a functional configuration of the information processing apparatus according to the embodiment of the disclosed technology;

FIG. 5 is a diagram showing an example of a structure of a data file generated by an association processing unit according to the embodiment of the disclosed technology;

FIG. 6 is a diagram showing an example of a playback screen according to the embodiment of the disclosed technology;

FIG. 7 is a flowchart showing an example of a flow of processing performed by a CPU according to the embodiment of the disclosed technology executing an association program;

FIG. 8 is a functional block diagram showing an example of a functional configuration of an information processing apparatus according to another embodiment of the disclosed technology;

FIG. 9 is a diagram showing an example of a playback screen according to another embodiment of the disclosed technology;

FIG. 10 is a functional block diagram showing an example of a functional configuration of an information processing apparatus according to still another embodiment of the disclosed technology;

FIG. 11A is a diagram showing an example of image extraction processing according to the embodiment of the disclosed technology;

FIG. 11B is a diagram showing an example of image extraction processing according to the embodiment of the disclosed technology;

FIG. 12 is a functional block diagram showing an example of a functional configuration of an information processing apparatus according to still another embodiment of the disclosed technology; and

FIG. 13 is a diagram showing an example of association processing and image extraction processing according to the embodiment of the disclosed technology.

### DETAILED DESCRIPTION

An example of embodiments of the disclosed technology will be described below with reference to the drawings. In addition, the same or equivalent components and parts in each drawing are given the same reference numerals, and duplicated descriptions will be omitted.

### First Embodiment

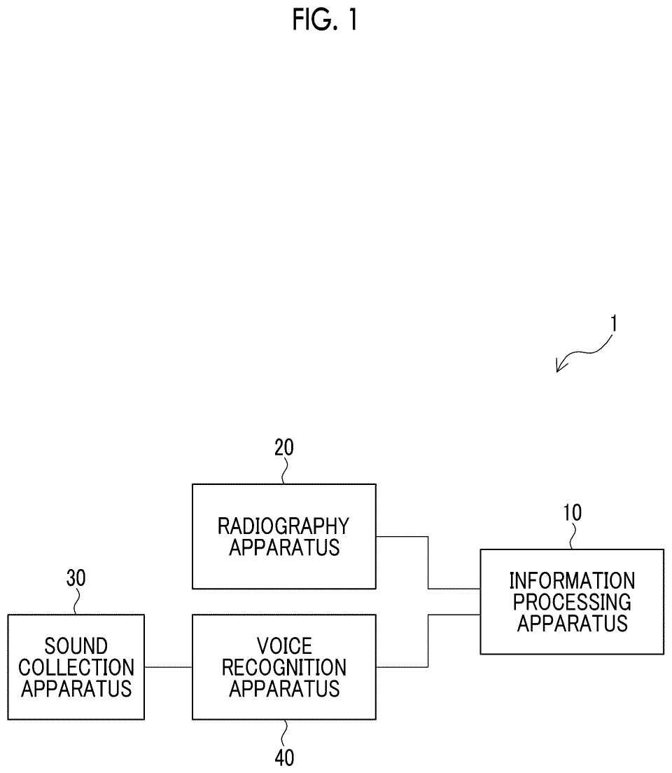

FIG. 1 is a diagram showing an example of a configuration of a radiography system 1 according to an embodiment of the disclosed technology. In the following description, an example in which the radiography system 1 is used for a videofluoroscopic examination of swallowing will be described. The radiography system 1 includes an information processing apparatus 10, a radiography apparatus 20, a sound collection apparatus 30, and a voice recognition apparatus 40.

The radiography apparatus 20 is capable of imaging a radiation image using radiation such as X-rays. It is also possible to sequentially display a series of radiation images captured by performing continuous irradiation with radiation in a time series manner, thereby producing a video display. The imaging of a series of radiation images for producing a video display is called fluoroscopy. In a videofluoroscopic examination of swallowing, fluoroscopy is performed to observe the process in which an examinee swallows a sample. Note that “continuous irradiation with radiation” includes both a case in which irradiation with radiation is continuously performed over a certain period of time and a case in which irradiation with pulsed radiation is performed a plurality of times within a certain period of time. The radiation image captured by the radiography apparatus 20 is immediately transmitted to the information processing apparatus 10.

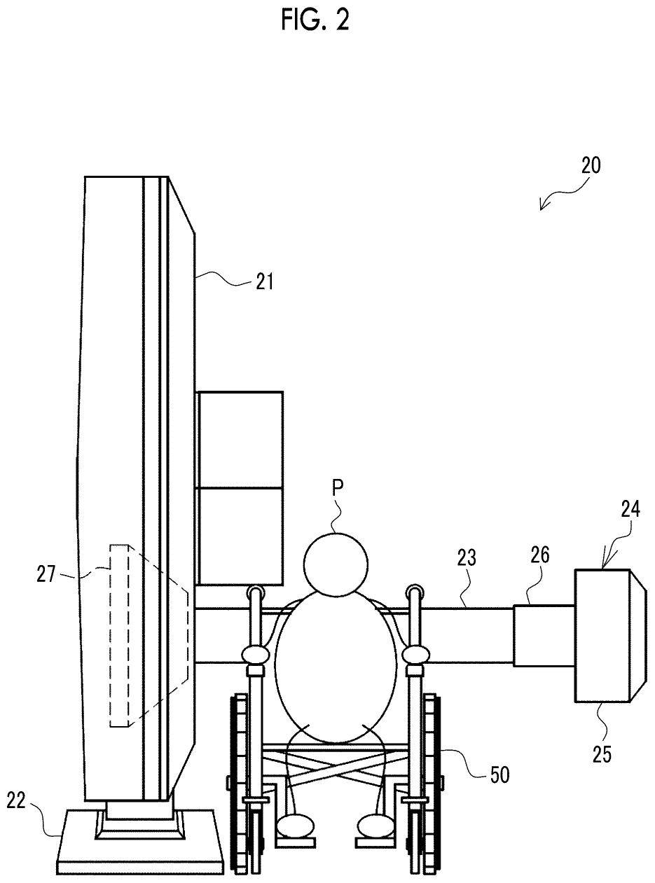

FIG. 2 is a diagram showing an example of the configuration of the radiography apparatus 20. The radiography apparatus 20 has an imaging table 21. The imaging table 21 is supported on the floor surface by a stand 22. A radiation generation unit 24 is attached to the imaging table 21 via a support column 23. The radiation generation unit 24 includes a radiation source 25 and a collimator 26. The imaging table 21 has a built-in radiation detector 27.

The radiation source 25 has a radiation tube (not shown) and irradiates an examinee P with radiation. The collimator 26 limits the irradiation field of radiation emitted from the radiation tube. The radiation detector 27 has a plurality of pixels that generate signal charges according to the radiation that has transmitted through the examinee P. The radiation detector 27 is called a flat panel detector (FPD).

The radiation generation unit 24 is capable of reciprocating together with the support column 23 along a long side direction of the imaging table 21 by a moving mechanism (not shown) such as a motor. The radiation detector 27 is capable of reciprocating along the long side direction of the imaging table 21 in conjunction with the movement of the radiation generation unit 24. The imaging table 21 and the support column 23 can be rotated between a standing state shown in FIG. 2 and a supine state (not shown) by a rotation mechanism (not shown) such as a motor. In the supine state, the surface of the imaging table 21 is parallel to the floor surface, and the direction in which the support column 23 extends is perpendicular to the floor surface. On the other hand, in the standing state, the surface of the imaging table 21 is perpendicular to the floor surface, and the direction in which the support column 23 extends is parallel to the floor surface. In the standing state, it is possible to perform fluoroscopy on the examinee P in a wheelchair 50 as shown in FIG. 2.

The sound collection apparatus 30 is a microphone that collects surrounding sounds. The uttered voice of an imaging person who captures a radiation image, such as a doctor or a radiologist, or the biological sound of an examinee is collected by the sound collection apparatus 30. The voice recognition apparatus 40 recognizes the voice collected by the sound collection apparatus 30 using a known voice recognition technology.

The voice recognition apparatus 40 generates a trigger signal in a case in which the collected voice is a specific voice. The trigger signal is a signal indicating a start point in time or an end point in time of a period of interest in the videofluoroscopic examination of swallowing. The specific voice may be an uttered voice including a specific word. For example, in a case in which the timing at which the examinee swallows the sample is set as the start point in time of the period of interest, the voice recognition apparatus 40 may generate a trigger signal in a case in which a word instructing the examinee to swallow the sample is included in the collected uttered voice. In a case in which a doctor or a radiologist instructs an examinee to swallow a sample, the doctor or the radiologist speaks an utterance including a word such as “please swallow”. The voice recognition apparatus 40 may generate a trigger signal in a case in which a specific word such as “swallow” is included in the uttered voice. The specific voice may also be a specific biological sound. For example, the voice recognition apparatus 40 may generate a trigger signal in a case in which the collected voice includes a swallowing sound uttered by the examinee swallowing a sample.

In a case in which the recognized voice includes a specific voice, the voice recognition apparatus 40 instantly generates a trigger signal and immediately transmits the trigger signal to the information processing apparatus 10. That is, the trigger signal is transmitted to the information processing apparatus 10 immediately after the occurrence of a specific voice. In a case in which a specific voice is intermittently uttered a plurality of times, the voice recognition apparatus 40 generates trigger signals corresponding to each of the specific voices in sequence, and transmits these trigger signals to the information processing apparatus 10 in sequence.

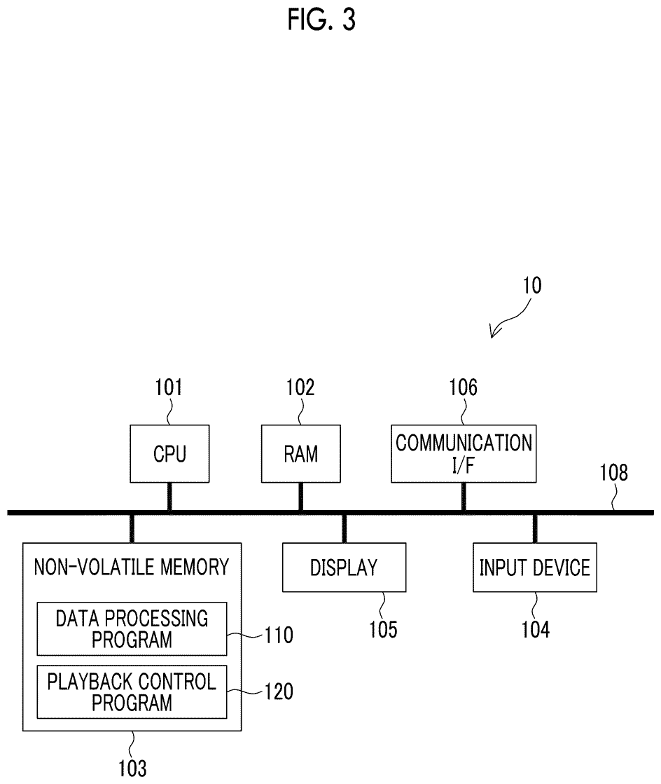

FIG. 3 is a diagram showing an example of a hardware configuration of the information processing apparatus 10. The information processing apparatus 10 includes a central processing unit (CPU) 101, a random-access memory (RAM) 102, a non-volatile memory 103, an input device 104 including a keyboard and a mouse, a display 105, and a communication interface 106. These pieces of hardware are connected to a bus 108.

The display 105 may be a touch panel display. The communication interface 106 is an interface for the information processing apparatus 10 to communicate with the radiography apparatus 20 and the voice recognition apparatus 40. The communication method may be either wired or wireless. For the wireless communication, it is possible to apply a method conforming to existing wireless communication standards such as, for example, Wi-Fi (registered trademark) and Bluetooth (registered trademark).

The non-volatile memory 103 is a non-volatile storage medium such as a hard disk or a flash memory. The non-volatile memory 103 stores a data processing program 110 and a playback control program 120. The RAM 102 is a work memory for the CPU 101 to execute processing. The CPU 101 loads the data processing program 110 or the playback control program 120 stored in the non-volatile memory 103 into the RAM 102, and executes processing in accordance with the data processing program 110 or the playback control program 120. The CPU 101 is an example of a “processor” in the disclosed technology.

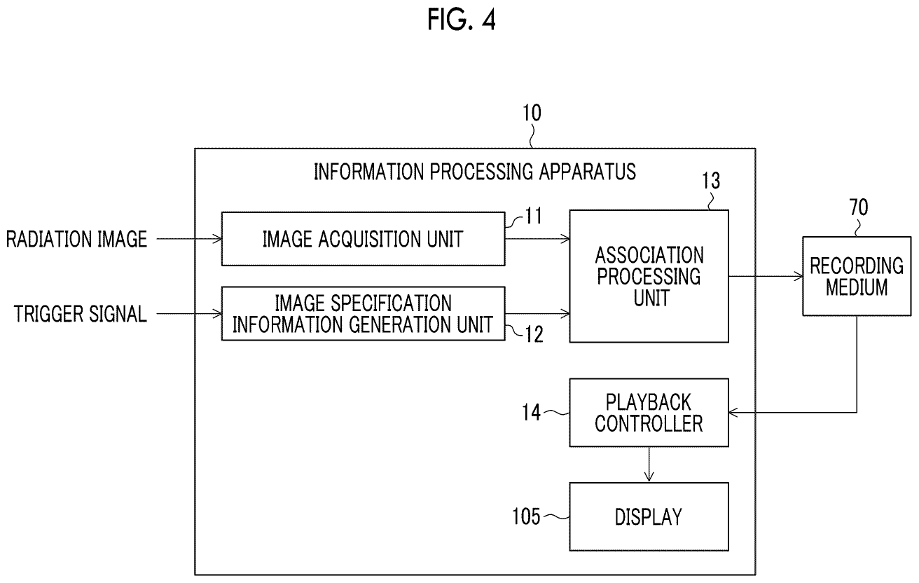

FIG. 4 is a functional block diagram showing an example of a functional configuration of the information processing apparatus 10. The information processing apparatus 10 includes an image acquisition unit 11, an image specification information generation unit 12, an association processing unit 13, and a playback controller 14. In a case in which the CPU 101 executes the data processing program 110, the information processing apparatus 10 functions as the image acquisition unit 11, the image acquisition unit 11, the image specification information generation unit 12, and the association processing unit 13. In a case in which the CPU 101 executes the playback control program 120, the information processing apparatus 10 functions as the playback controller 14.

The image acquisition unit 11 acquires a series of radiation images captured by performing continuous irradiation with radiation in the radiography apparatus 20. A series of radiation images are obtained by fluoroscopically imaging the process in which an examinec chews and swallows a sample in a videofluoroscopic examination of swallowing.

The image specification information generation unit 12 generates image specification information. The image specification information is information for specifying a radiation image selected from among a series of radiation images according to a timing of occurrence of a specific voice uttered during the imaging period of the radiation images. The image specification information generation unit 12 recognizes the timing of occurrence of a specific voice through a trigger signal transmitted from the voice recognition apparatus 40. The image specification information generation unit 12 selects the radiation image acquired at a point in time of reception of the trigger signal as an image (keyframe) at the start point in time or the end point in time of the period of interest from among the series of radiation images acquired by the image acquisition unit 11. The image specification information generation unit 12 generates image specification information for specifying the selected image (keyframe). For example, the image specification information generation unit 12 may generate information (that is, a frame number) indicating which radiation image (keyframe) is acquired at the point in time of reception of the trigger signal as image specification information. In addition, the image specification information generation unit 12 may generate information indicating an clapsed time from the start of acquisition of the series of radiation images to the point in time of reception of the trigger signal as image specification information. The image specification information generation unit 12 generates a plurality of pieces of image specification information based on each of a plurality of trigger signals in a case in which the plurality of trigger signals are received intermittently during the imaging period of a series of radiation images.

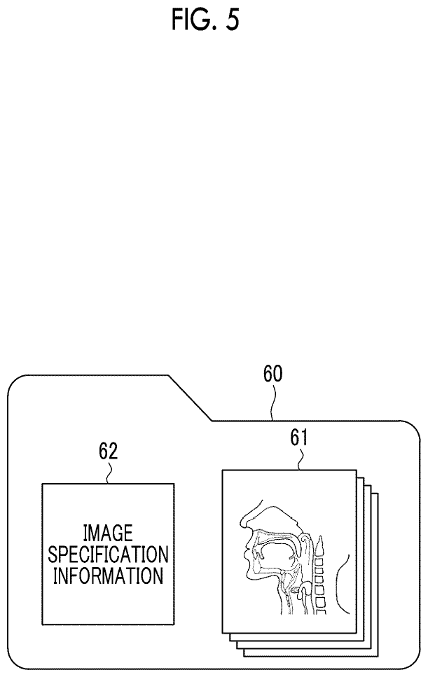

The association processing unit 13 associates the series of radiation images acquired by the image acquisition unit 11 with the image specification information generated by the image specification information generation unit 12. For example, the association processing unit 13 may associate a series of radiation images with the image specification information by generating a data file 60 including image specification information 62 and image group data 61 including a series of radiation images, as shown in FIG. 5. In a case in which a plurality of pieces of image specification information are generated based on a plurality of voices uttered intermittently during the imaging period of the series of radiation images, the association processing unit 13 associates the plurality of pieces of image specification information with the series of radiation images.

The association processing unit 13 stores a data file in which the series of radiation images and the image specification information are associated with each other in a recording medium 70. The recording medium 70 in which the data file is stored may be the non-volatile memory 103 included in the information processing apparatus 10, or may be a recording medium included in an external image server (not shown).

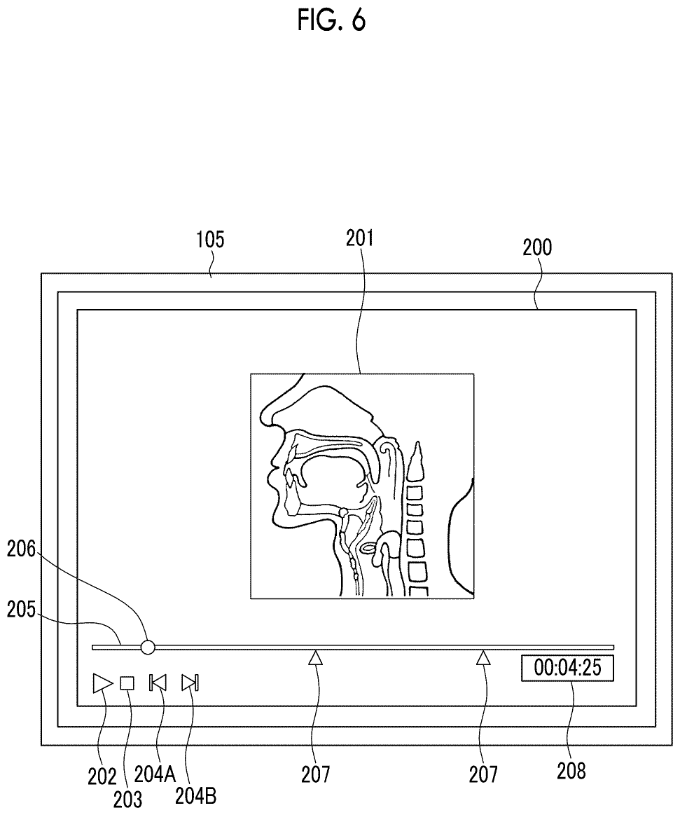

The playback controller 14 reads out a data file stored in the recording medium 70 and performs playback control to display a series of radiation images included in the data file as a video. A video composed of a series of radiation images will be hereinafter referred to as an examination video. The playback controller 14 displays a playback screen 200 illustrated in FIG. 6 on the display 105 and displays an examination video 201 on the playback screen 200.

On the playback screen 200, a play/pause button 202, a stop button 203, skip buttons 204A and 204B, a seek bar 205, a slider 206, a keyframe mark 207, and a playback time 208 are displayed together with the examination video 201. The play/pause button 202 is an operation input unit for performing playback and pause of the examination video 201. The stop button 203 is an operation input unit for stopping the playback of the examination video 201. The seck bar 205 and the slider 206 indicate a current playback position of the examination video 201. The user can change the playback position of the examination video 201 by sliding the slider 206 on the seek bar 205.

The keyframe mark 207 indicates a time position of the radiation image (keyframe) specified by the image specification information in the examination video 201. The position on the seek bar 205 indicated by the keyframe mark 207 is a time position of the keyframe in the examination video 201. In FIG. 6, there are two radiation images (keyframes) specified by the image specification information, and two keyframe marks 207 corresponding to the two keyframes are displayed.

The skip buttons 204A and 204B are operation input units for moving the playback position of the examination video 201 to the position of the radiation image (keyframe) specified by the image specification information. In a case in which the skip button 204A is pressed, the playback controller 14 moves the playback position of the examination video 201 to the position of the next keyframe mark 207. In a case in which the skip button 204B is pressed, the playback controller 14 moves the playback position of the examination video 201 to the position of the previous keyframe mark 207. The playback controller 14 can specify keyframes in the examination video 201 by referring to the image specification information.

The user can change the time position of the keyframe in the examination video 201 by changing the position on the seek bar 205 indicated by the keyframe mark 207. The image specification information is changed in association with the change of the time position of the keyframe in the examination video 201. That is, the image specification information generation unit 12 changes the image specification information based on the change operation of the time position of the keyframe specified by the image specification information in the examination video 201. Accordingly, for example, in a case in which the time position of the radiation image specified by the image specification information is deviated due to a deviation in the timing of occurrence of the trigger signal or the like, the deviation can be corrected.

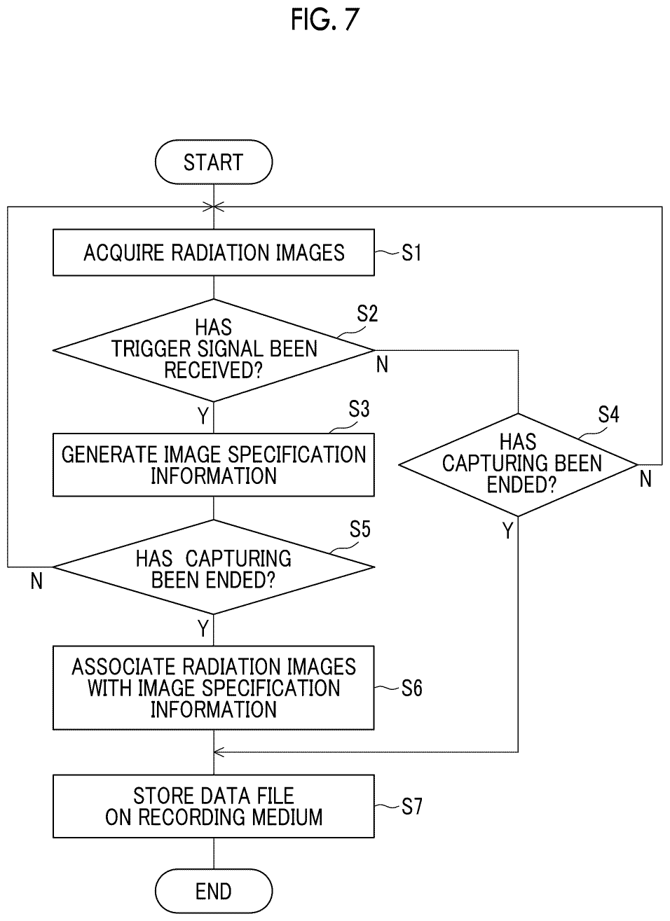

FIG. 7 is a flowchart showing an example of a flow of processing performed by the CPU 101 executing the data processing program 110. In Step S1, the image acquisition unit 11 acquires a series of radiation images captured by performing continuous irradiation with radiation in the radiography apparatus 20. A series of radiation images are obtained by fluoroscopically imaging the process in which an examince swallows a sample in a videofluoroscopic examination of swallowing. The acquisition of a series of radiation images continues until the irradiation of radiation (fluoroscopy) ends in the radiography apparatus 20.

During the imaging period of the radiation image, the voice recognition apparatus 40 generates a trigger signal in a case in which the voice collected by the sound collection apparatus 30 includes a specific voice, and transmits the trigger signal to the information processing apparatus 10.

In Step S2, the image specification information generation unit 12 determines whether or not the trigger signal has been received. In a case in which it is determined that the trigger signal has been received, the process proceeds to Step S3, and in a case in which it is determined that the trigger signal has not been received, the process proceeds to Step S4.

In Step S3, the image specification information generation unit 12 selects the radiation image acquired at the point in time of reception of the trigger signal as an image (keyframe) at the start point in time or the end point in time of the period of interest, and generates image specification information for specifying the selected image (keyframe).

In Step S4, the CPU 101 determines whether or not capturing of the radiation image has been ended. The determination as to whether or not the capturing of the radiation image has been ended can be performed based on, for example, a status signal supplied from the radiography apparatus 20. In a case in which it is determined that the capturing of the radiation image has been ended, the process proceeds to Step S7, and in a case in which it is determined that the capturing of the radiation image has not been ended, the process returns to Step S1.

In Step S5, the CPU 101 determines whether or not the capturing of the radiation image has been ended, as in Step S4. In a case in which it is determined that the capturing of the radiation image has been ended, the process proceeds to Step S6, and in a case in which it is determined that the capturing of the radiation image has not been ended, the process returns to Step S1.

In Step S6, the association processing unit 13 associates the series of radiation images acquired in Step S1 with the image specification information generated in Step S3. The association processing unit 13 may associate a series of radiation images with the image specification information, for example, by generating a data file 60 including image specification information 62 and image group data 61 including a series of radiation images, as shown in FIG. 5.

In Step S7, the association processing unit 13 stores a data file in which the series of radiation images and the image specification information are associated with each other in the recording medium 70.

As described above, the information processing apparatus 10 according to the embodiment of the disclosed technology acquires a series of radiation images captured by performing continuous irradiation with radiation, generates, in a case in which a voice uttered during an imaging period of the series of radiation images is a specific voice, image specification information for specifying a radiation image selected from among the series of radiation images according to a timing of occurrence of the voice, and associates the image specification information with the series of radiation images.

In a videofluoroscopic examination of swallowing, radiation images captured in a period of interest required for diagnosis are the main target of observation. However, the series of radiation images captured in a videofluoroscopic examination of swallowing may also include images captured during periods other than the period of interest. In this case, it is necessary to search for the images during the period of interest from among a series of captured radiation images, which places a heavy burden on the observer.

With the information processing apparatus 10 according to the present embodiment, information for specifying the image (keyframe) at the start point in time or the end point in time of the period of interest among the series of the radiation images can be generated as image specification information. The image specification information is then associated with a series of radiation images. Accordingly, in the playback of the video (examination video) composed of a series of radiation images, it is possible to easily extract an image (keyframe) specified by the image specification information. For example, by operating the skip buttons 204A and 204B (see FIG. 6), it is possible to move the playback position of the examination video to the keyframe. Accordingly, it is not necessary to search for images during a period of interest from among a series of the radiation images, thereby reducing the burden on the observer. That is, with the information processing apparatus 10 according to the present embodiment, it is possible to easily access a desired image from a series of radiation images.

In addition, the image specification information is generated based on a trigger signal transmitted from the voice recognition apparatus 40. The voice recognition apparatus 40 generates a trigger signal in a case in which the voice collected by the sound collection apparatus 30 is a specific voice. Accordingly, for example, it is possible to generate the image specification information based on the utterance of a doctor, a radiologist, or the like. That is, since it is possible to select an image (keyframe) at the start point in time or the end point in time of the period of interest by voice, it is not necessary for the user to perform a special operation for selecting the keyframe.

In a videofluoroscopic examination of swallowing, radiation images may be continuously captured while changing the combination of the examinee's posture and the type of sample (that is, while changing the examination level). In this case, a plurality of examination levels are mixed in one examination video, and it is thus difficult to extract a portion related to a desired examination level from the examination video.

With the information processing apparatus 10 according to the present embodiment, image specification information for specifying each of the radiation images acquired at the timing of transition from one examination level to the next examination level is generated and associated with the series of radiation images, so that even in a case in which a plurality of examination levels are mixed in one examination video, it is easy to extract a portion related to each examination level from the series of radiation images. For example, in a case in which a doctor or a radiologist makes an utterance indicating the type of examination level to be performed in the transition of the examination levels, the voice recognition apparatus 40 is configured to generate a trigger signal in response to a word indicating the type of examination level included in the uttered voice, so that it is possible to generate image specification information for specifying the radiation image acquired at the timing of the transition of the examination level.

### Second Embodiment

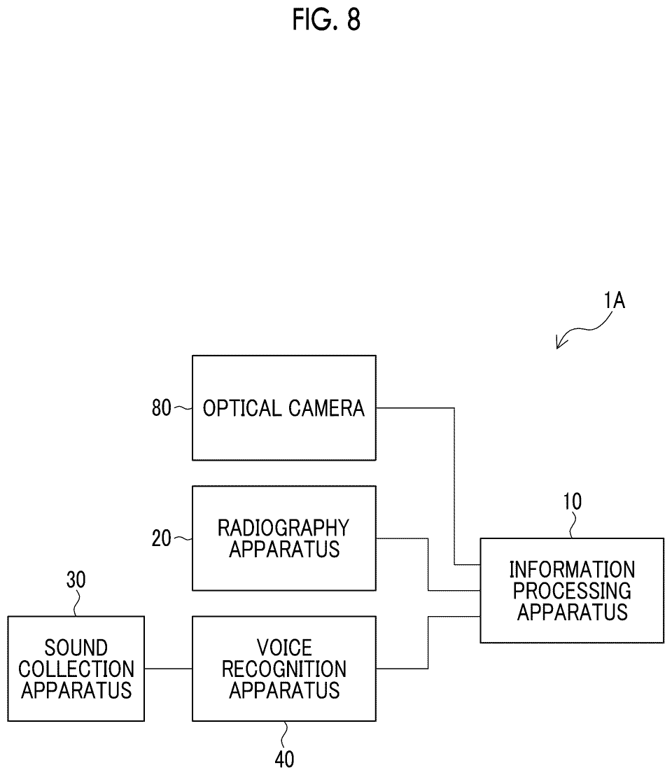

FIG. 8 is a diagram showing an example of a configuration of a radiography system 1A according to a second embodiment of the disclosed technology. The radiography system 1A differs from the radiography system 1 (see FIG. 1) according to the first embodiment in that the radiography system 1A further includes an optical camera 80.

The optical camera 80 is a digital video camera that captures optical images. In parallel with the capturing of a series of radiation images by the radiography apparatus 20, the capturing of a series of optical images by the optical camera 80 is carried out. The optical image captured by the optical camera 80 is immediately transmitted to the information processing apparatus 10.

The image acquisition unit 11 acquires a series of radiation images captured by performing continuous irradiation with radiation in the radiography apparatus 20. A series of radiation images are obtained by fluoroscopically imaging the process in which an examinec chews and swallows a sample in a videofluoroscopic examination of swallowing. The image acquisition unit 11 further acquires a series of optical images continuously captured in parallel with the capturing of the series of radiation images. A series of optical images are optical captures of the process in which an examince chews and swallows a sample in a videofluoroscopic examination of swallowing.

The image specification information generation unit 12 generates first image specification information and second image specification information. The first image specification information is information for specifying a radiation image selected from among a series of radiation images according to a timing of occurrence of a specific voice uttered during the imaging period of the radiation images. The second image specification information is information for specifying an optical image selected from among a series of optical images according to a timing of occurrence of a specific voice uttered during the imaging period of the optical images.

The image specification information generation unit 12 recognizes the timing of occurrence of a specific voice through a trigger signal transmitted from the voice recognition apparatus 40. The image specification information generation unit 12 selects the radiation image acquired at a point in time of reception of the trigger signal as a radiation image (keyframe) at the start point in time or the end point in time of the period of interest from among the series of radiation images acquired by the image acquisition unit 11. The image specification information generation unit 12 generates first image specification information for specifying the selected radiation image (keyframe). Similarly, the image specification information generation unit 12 selects the optical image acquired at a point in time of reception of the trigger signal as an optical image (keyframe) at the start point in time or the end point in time of the period of interest from among the series of optical images acquired by the image acquisition unit 11. The image specification information generation unit 12 generates second image specification information for specifying the selected optical image (keyframe). The first image specification information and the second image specification information are generated based on a common trigger signal. That is, the radiation image specified by the first image specification information and the optical image specified by the second image specification information are images captured at the same time point.

The association processing unit 13 associates the series of radiation images acquired by the image acquisition unit 11 with the first image specification information generated by the image specification information generation unit 12. Furthermore, the association processing unit 13 associates the series of optical images acquired by the image acquisition unit 11 with the second image specification information generated by the image specification information generation unit 12. In a case in which a plurality of pieces of first image specification information and a plurality of pieces of second image specification information are generated based on a plurality of voices uttered intermittently during the imaging period of the series of radiation images and a series of optical images, the association processing unit 13 associates the plurality of pieces of first image specification information with the series of radiation images and associates the plurality of pieces of second image specification information with the series of optical images.

The association processing unit 13 stores a data file in which the series of radiation images and the first image specification information are associated with each other in the recording medium 70. Furthermore, the association processing unit 13 stores a data file in which the series of optical images and the second image specification information are associated with each other in the recording medium 70.

The playback controller 14 reads out the data file stored in the recording medium 70 and performs playback control of a radiation video (hereinafter referred to as a first examination video) composed of a series of radiation images and an optical video (hereinafter referred to as a second examination video) composed of a series of optical images included in the data file.

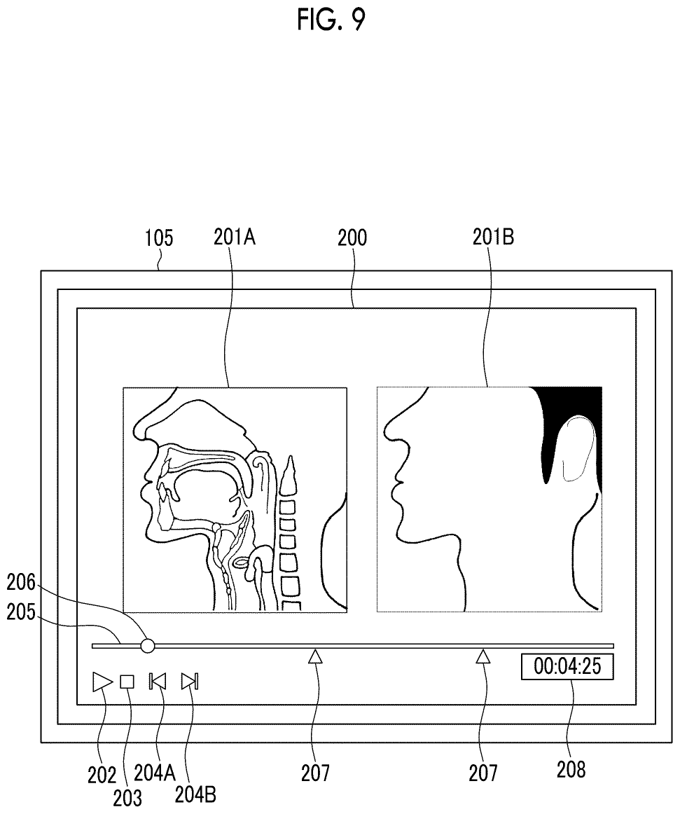

The playback controller 14 displays a playback screen 200 illustrated in FIG. 9 on the display 105 and displays a first examination video 201A and a second examination video 201B simultaneously on the playback screen 200. The playback controller 14 plays back the first examination video 201A and the second examination video 201B such that images at the same time point are displayed.

The keyframe mark 207 indicates time positions of the radiation images and the optical images (keyframes) specified by the first image specification information and the second image specification information, respectively, in the first examination video 201A and the second examination video 201B. The position on the seek bar 205 indicated by the keyframe mark 207 is a time position of the keyframe in the examination video.

The skip buttons 204A and 204B are operation input units for moving the playback position of the first examination video 201A and the second examination video 201B to the position of the radiation image (keyframe) specified by the first image specification information and the second image specification information. In a case in which the skip button 204A is pressed, the playback controller 14 moves the playback position of the first examination video 201A and the second examination video 201B to the position of the next keyframe mark 207, respectively. In a case in which the skip button 204B is pressed, the playback controller 14 moves the playback position of the first examination video 201A and the second examination video 201B to the position of the previous keyframe mark 207, respectively. The playback controller 14 can specify keyframes in the first examination video 201A and the second examination video 201B by referring to the first image specification information and the second image specification information.

As described above, the information processing apparatus 10 according to the second embodiment of the disclosed technology acquires a series of optical images that are continuously captured in parallel with the capturing of the series of radiation images, and plays back each of a radiation video composed of the series of radiation images and an optical video composed of the series of optical images such that images at the same time point are displayed.

In a videofluoroscopic examination of swallowing, it may be difficult to ascertain the progress state of the examination, the state of the examinee, and the like only from the radiation images. By playing back the radiation video and the optical video are played back such that the images at the same time point, it becomes easier to ascertain the progress state of the examination, the state of the examinee, and the like.

### Third Embodiment

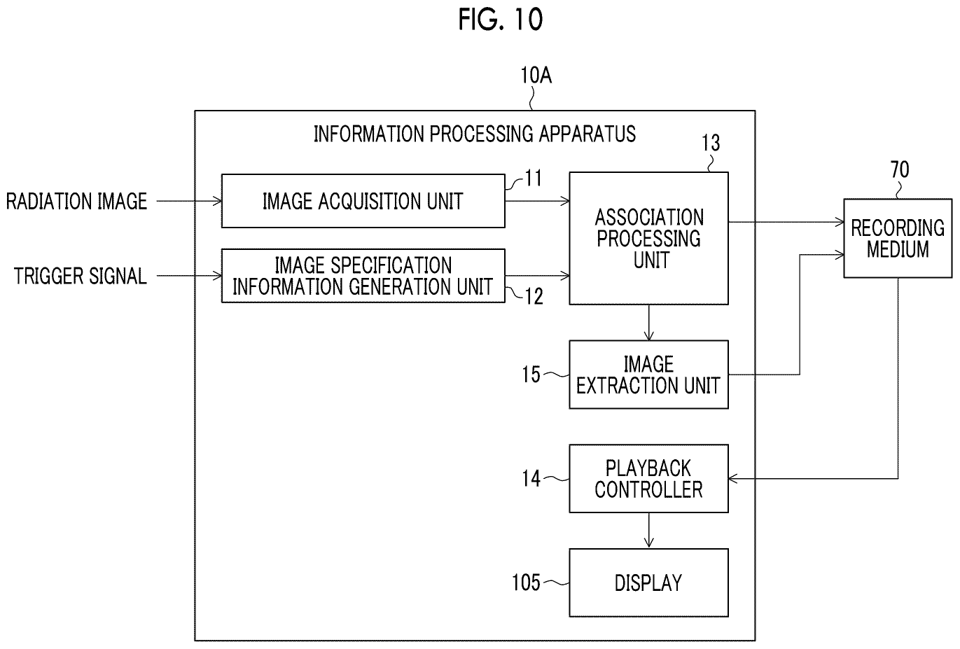

FIG. 10 is a functional block diagram showing an example of a functional configuration of an information processing apparatus 10A according to a third embodiment of the disclosed technology. The information processing apparatus 10A differs from the information processing apparatus 10 (see FIG. 4) according to the first embodiment in that the information processing apparatus 10A further includes an image extraction unit 15.

The image extraction unit 15 extracts some radiation images from a series of radiation images with a radiation image specified by the image specification information as a starting point. The image extraction unit 15 generates a data file including the extracted some radiation images.

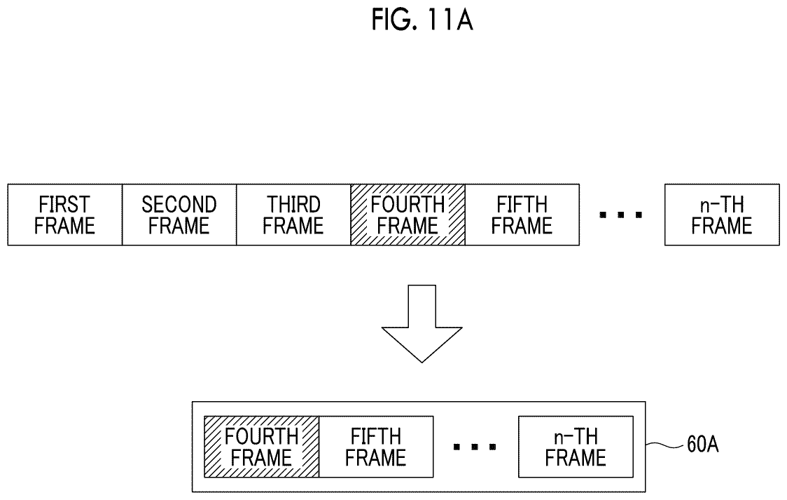

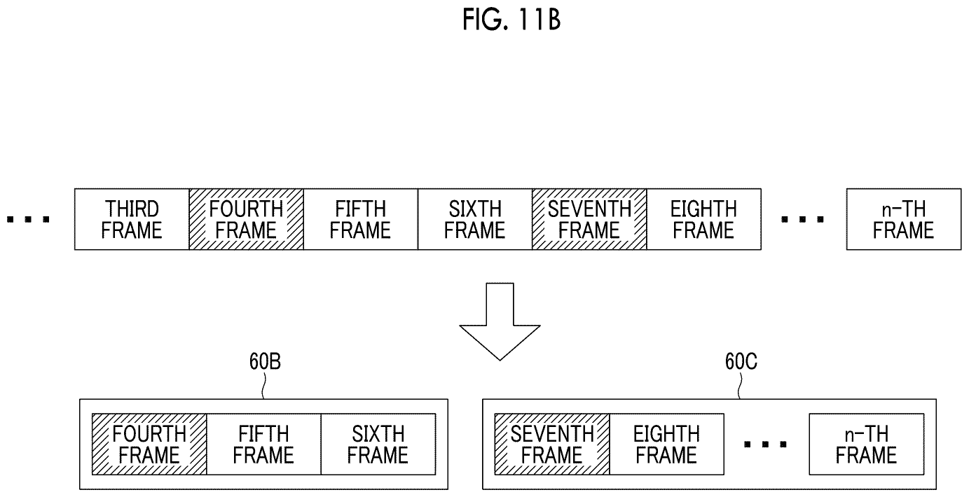

FIG. 11A is a diagram showing an example of image extraction processing by the image extraction unit 15. A series of radiation images acquired by the image acquisition unit 11 constitute each frame of the examination video. FIG. 11A illustrates a case in which the image acquisition unit 11 acquires a series of radiation images corresponding to the first frame to the n-th frame of the examination video. The fourth frame, which is hatched in FIG. 11A, is an image (keyframe) specified by the image specification information.

The image extraction unit 15 extracts some radiation images with the fourth frame specified by the image specification information as a starting point from a series of the radiation images corresponding to the first frame to the n-th frame of the examination video. FIG. 11A illustrates a case in which radiation images corresponding to the fourth frame to the n-th frame are extracted. The image extraction unit 15 generates a data file 60A including the extracted radiation images corresponding to the fourth frame to the n-th frame. The image extraction unit 15 stores the generated data file 60A in the recording medium 70.

An original data file including a series of radiation images corresponding to the first frame to the n-th frame of the examination video, and the data file generated by the image extraction unit 15 including the radiation images corresponding to the fourth frame to the n-th frame are stored in the recording medium 70, respectively.

FIG. 11B is a diagram showing another example of image extraction processing by the image extraction unit 15. The fourth frame and the seventh frame, which are hatched in FIG. 11B, are images (keyframes) specified by the image specification information.

The image extraction unit 15 extracts some radiation images with the fourth frame and the seventh frame specified by the image specification information as starting points, respectively, from a series of the radiation images corresponding to the first frame to the n-th frame of the examination video. FIG. 11B illustrates a case in which radiation images corresponding to the fourth frame to the sixth frame and radiation images corresponding to the seventh frame to the n-th frame are extracted. The image extraction unit 15 generates a data file 60B including the extracted radiation images corresponding to the fourth frame to the sixth frame and a data file 60C including the extracted radiation images corresponding to the seventh frame to the n-th frame. The image extraction unit 15 stores the generated data files 60B and 60C in the recording medium 70, respectively.

The playback controller 14 reads out the data files stored in the recording medium 70 and performs playback control of the video composed of the radiation images included in the data files. The data file to be played back can be designated by the user. For example, in a case in which the data file 60C shown in FIG. 11B is designated as the file to be played back, the examination video composed of the seventh frame to the n-th frame is played back.

As described above, the information processing apparatus 10A according to the third embodiment of the disclosed technology extracts some radiation images from a series of radiation images with a radiation image specified by image specification information as a starting point, and generates a data file including the extracted some radiation images. With the information processing apparatus 10A according to the present embodiment, for example, by configuring the image specification information to specify a radiation image at the start point in time of a period of interest, it is possible to generate a data file in which images during the period of interest are extracted from a series of captured radiation images. Accordingly, it is not necessary to search for images during a period of interest from among a series of the radiation images, thereby reducing the burden on the user. That is, with the information processing apparatus 10A according to the present embodiment, it is possible to easily access a desired image from a series of radiation images.

Note that, in the above description, the case in which some radiation images are extracted from a series of radiation images with a radiation image specified by image specification information as a starting point has been described as an example, but some radiation images may be extracted from a series of radiation images with a radiation image specified by image specification information as an ending point.

### Fourth Embodiment

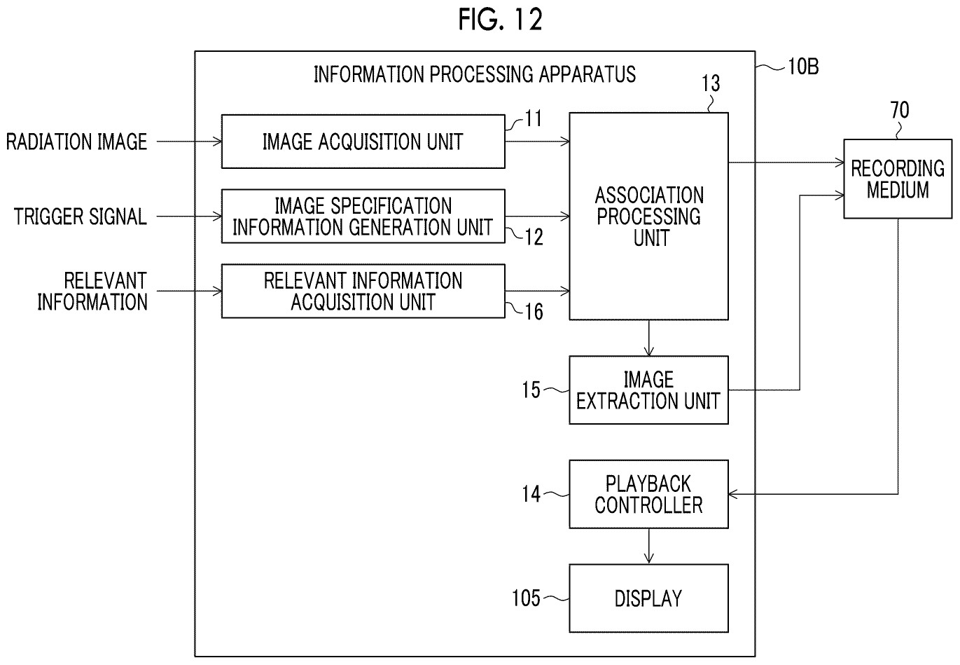

FIG. 12 is a functional block diagram showing an example of a functional configuration of an information processing apparatus 10B according to a fourth embodiment of the disclosed technology. The information processing apparatus 10B differs from the information processing apparatus 10A (see FIG. 10) according to the third embodiment in that the information processing apparatus 10B further includes a relevant information acquisition unit 16.

In the present embodiment, in a case in which the collected voice includes a predetermined specific term indicating the content of the examination level in the videofluoroscopic examination of swallowing, the voice recognition apparatus 40 generates identification information of the examination level indicated by the term as relevant information related to the imaging of the radiation image.

Specific terms indicating the content of the examination level may include terms indicating the type of sample that the examinee swallows in the videofluoroscopic examination of swallowing, such as “water”, “thick liquid”, “chopped food”, and “porridge”. In this case, the relevant information includes identification information of the type of sample. In addition, specific terms indicating the content of the examination level may include terms indicating the type of posture taken by the examinee during the videofluoroscopic examination of swallowing, such as “45°”, “supine”, and “prone”. In this case, the relevant information includes identification information of the type of posture of the examinee. In addition, the specific terms indicating the content of the examination level may include terms indicating the type of imaging direction of the radiation images captured in the videofluoroscopic examination of swallowing, such as “frontal imaging” and “lateral imaging”. In this case, the relevant information includes identification information for the type of imaging direction.

For example, in a case in which the voice collected by the sound collection apparatus 30 includes the term “thick liquid”, which indicates the content of the examination level, the voice recognition apparatus 40 generates identification information corresponding to “thick liquid” as relevant information. In this case, the voice recognition apparatus 40 may generate, as relevant information, for example, text describing “thick liquid” or a level code consisting of characters, numerical values, symbols, or a combination of these corresponding to “thick liquid”. In a case in which the recognized voice includes a specific term, the voice recognition apparatus 40 instantly generates relevant information and immediately transmits the relevant information to the information processing apparatus 10B. That is, the relevant information is transmitted to the information processing apparatus 10B immediately after the occurrence of a voice including a specific term. In a case in which an utterance including a specific term is made intermittently over a plurality of times, relevant information corresponding to each utterance is generated in sequence and transmitted to the information processing apparatus 10B in sequence.

The relevant information acquisition unit 16 acquires relevant information generated by the voice recognition apparatus 40. The relevant information is information related to the imaging content of the series of radiation images acquired by the image acquisition unit 11. Specifically, identification information of the examination level generated based on the utterance of a doctor or radiologist at the time of capturing a series of radiation images is acquired as relevant information. The relevant information is acquired immediately after the occurrence of a voice including a specific term. In a case in which a plurality of pieces of relevant information are generated intermittently, the relevant information is acquired by the relevant information acquisition unit 16 in sequence.

The association processing unit 13 associates the series of radiation images acquired by the image acquisition unit 11, the image specification information generated by the image specification information generation unit 12, and the relevant information acquired by the relevant information acquisition unit 16 with each other. Specifically, the association processing unit 13 associates the relevant information acquired within a predetermined period before and after the point in time of reception of the trigger signal with the radiation image (keyframe) specified by the image specification information generated in response to the trigger signal.

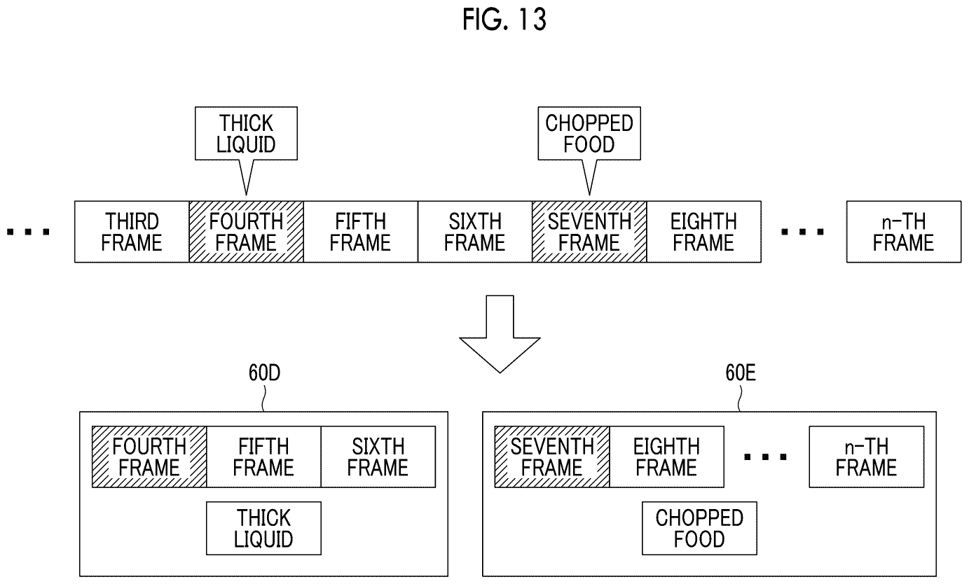

FIG. 13 is a diagram showing an example of association processing in the association processing unit 13 and image extraction processing by the image extraction unit 15. The fourth frame and the seventh frame, which are hatched in FIG. 13, are images (keyframes) specified by the image specification information. This means that the trigger signal is received at each of a point in time of acquisition of the fourth frame and a point in time of acquisition of the seventh frame. In the example shown in FIG. 13, relevant information corresponding to “thick liquid” is acquired within a predetermined period before and after the point in time of reception of the trigger signal corresponding to the fourth frame, and the relevant information is associated with the fourth frame. Further, relevant information corresponding to “chopped food” is acquired within a predetermined period before and after the point in time of reception of the trigger signal corresponding to the seventh frame, and the relevant information is associated with the seventh frame.

The image extraction unit 15 generates a data file 60D in which the radiation images constituting the fourth frame to the sixth frame and the relevant information corresponding to “thick liquid” are associated with each other. Furthermore, the image extraction unit 15 generates a data file 60E in which the radiation images constituting the seventh frame to the n-th frame and the relevant information corresponding to “chopped food” are associated with each other. The image extraction unit 15 stores the generated data files 60D and 60E in the recording medium 70, respectively.

As described above, the information processing apparatus 10B according to the fourth embodiment of the disclosed technology extracts some radiation images from a series of radiation images with a radiation image specified by image specification information as a starting point or an ending point, similarly to the third embodiment. Further, the information processing apparatus 10B acquires relevant information related to the imaging content of a series of radiation images, and generates a data file in which the relevant information and some radiation images extracted from the series of radiation images are associated with each other.

In a videofluoroscopic examination of swallowing, radiation images may be continuously captured while changing the combination of the examinee's posture and the type of sample (that is, while changing the examination level), and a plurality of examination levels may be mixed in one examination video. In this case, it is necessary to accurately associate the content of the examination level with the frame related to the examination level such that it is possible to ascertain which portion of the series of radiation images corresponds to which examination level.

With the information processing apparatus 10B according to the present embodiment, by configuring the image specification information to specify the radiation image at the point in time of transition of the examination level, it is possible to generate a plurality of data files corresponding to each of the plurality of examination levels. Further, it is possible to include relevant information indicating the content of the corresponding examination level in each of the plurality of data files. In addition, since the series of radiation images are divided for each examination level based on the recognized voice and the content of the examination level is associated with each divided image, it is possible to reduce the burden on the user.

In the present embodiment, a case in which the relevant information is generated by voice recognition has been described as an example, but the disclosed technology is not limited to this aspect. For example, the relevant information can also be generated by a function button operation or a keyboard operation.

In the above first to fourth embodiments, a case in which the disclosed technology is applied to a radiation image acquired in a videofluoroscopic examination of swallowing has been described as an example, but the disclosed technology is not limited to this aspect. The disclosed technology can be applied to any radiation images acquired in examinations, diagnoses, or the like other than videofluoroscopic examinations of swallowing.

In addition, in each of the above embodiments, a configuration in which the radiography system 1 or 1A comprises the voice recognition apparatus 40 has been described as an example, but the information processing apparatus 10, 10A, or 10B may have at least a part of the functions of the voice recognition apparatus 40. For example, the voice recognition apparatus 40 may transmit a voice recognition result to the information processing apparatus 10, 10A, or 10B and the information processing apparatus 10, 10A, or 10B may generate image specification information and relevant information based on the recognized voice. Furthermore, the information processing apparatuses 10, 10A, and 10B may recognize a voice and generate image specification information and relevant information based on the recognized voice.

As hardware for executing processes in each functional unit of the information processing apparatuses 10, 10A, and 10B, various processors as shown below can be used. The processor may be a CPU that executes software (programs) and functions as various processing units. Furthermore, the processor may be a programmable logic device (PLD) such as an FPGA whose circuit configuration is changeable. Moreover, the processor may have a circuit configuration that is specially designed to execute a specific process, such as an application-specific integrated circuit (ASIC).

Each functional unit of the information processing apparatuses 10, 10A, and 10B may be configured by one of the various processors described above, or may be configured by a combination of the same or different kinds of two or more processors (for example, a combination of a plurality of FPGAs or a combination of the CPU and the FPGA). In addition, a plurality of functional units may be configured by one processor.

In the above embodiments, the data processing program 110 and the playback control program 120 have been described as being stored (installed) in the non-volatile memory 103 in advance; however, the present disclosure is not limited thereto. The data processing program 110 and the playback control program 120 may be provided in a form recorded in a recording medium such as a compact disc read-only memory (CD-ROM), a digital versatile disc read-only memory (DVD-ROM), and a universal serial bus (USB) memory. In addition, the data processing program 110 and the playback control program 120 may be configured to be downloaded from an external device via a network.

Regarding the first to fourth embodiments, the following supplementary notes are further disclosed.

### Supplementary Note 1

An information processing apparatus comprising at least one processor,
in which the processor is configured to:
   acquire a series of radiation images captured by performing continuous irradiation with radiation;
   generate, in a case in which a voice uttered during an imaging period of the series of radiation images is a specific voice, image specification information for specifying a radiation image selected from among the series of radiation images according to a timing of occurrence of the voice; and
   associate the image specification information with the series of radiation images.

### Supplementary Note 2

The information processing apparatus according to Supplementary Note 1,
in which the specific voice is an uttered voice including a specific word.

### Supplementary Note 3

The information processing apparatus according to Supplementary Note 1 or 2,
in which the specific voice is a specific biological sound.

### Supplementary Note 4

The information processing apparatus according to any one of Supplementary Notes 1 to 3,
in which the processor is configured to:
   generate a plurality of pieces of the image specification information based on a plurality of voices uttered intermittently during the imaging period of the series of radiation images; and
   associate the plurality of pieces of image specification information with the series of radiation images.

### Supplementary Note 5

The information processing apparatus according to any one of Supplementary Notes 1 to 4,
in which the processor is configured to display a time position of the radiation image specified by the image specification information in a video composed of the series of radiation images.

### Supplementary Note 6

The information processing apparatus according to any one of Supplementary Notes 1 to 5,
in which the processor is configured to change the image specification information based on a change operation of a time position of the radiation image specified by the image specification information in a video composed of the series of radiation images.

### Supplementary Note 7

The information processing apparatus according to any one of Supplementary Notes 1 to 6,
in which the processor is configured to:
   acquire a series of optical images that are continuously captured in parallel with the capturing of the series of radiation images; and
   play back each of a radiation video composed of the series of radiation images and an optical video composed of the series of optical images such that images at the same time point are displayed.

### Supplementary Note 8

The information processing apparatus according to any one of Supplementary Notes 1 to 7,
in which the processor is configured to:
   extract some radiation images from the series of radiation images with the radiation image specified by the image specification information as a starting point or an ending point; and
   generate a data file including the extracted some radiation images.

### Supplementary Note 9

The information processing apparatus according to Supplementary Note 8,
in which the processor is configured to:
   acquire relevant information related to imaging content of the series of radiation images; and
   generate a data file in which the relevant information and the some radiation images are associated with each other.

### Supplementary Note 10

An information processing method executed by at least one processor included in an information processing apparatus, the information processing method comprising:
acquiring a series of radiation images captured by performing continuous irradiation with radiation;
generating, in a case in which a voice uttered during an imaging period of the series of radiation images is a specific voice, image specification information for specifying a radiation image selected from among the series of radiation images according to a timing of occurrence of the voice; and
associating the image specification information with the series of radiation images.

### Supplementary Note 11

A program for causing at least one processor included in an information processing apparatus to execute:
acquiring a series of radiation images captured by performing continuous irradiation with radiation;
generating, in a case in which a voice uttered during an imaging period of the series of radiation images is a specific voice, image specification information for specifying a radiation image selected from among the series of radiation images according to a timing of occurrence of the voice; and
associating the image specification information with the series of radiation images.

What is claimed is:

1. An information processing apparatus comprising at least one processor,
wherein the processor is configured to:
acquire a series of radiation images captured by performing continuous irradiation with radiation;
generate, in a case in which a voice uttered during an imaging period of the series of radiation images is a specific voice, image specification information for specifying a radiation image selected from among the series of radiation images according to a timing of occurrence of the voice; and
associate the image specification information with the series of radiation images.

2. The information processing apparatus according to claim 1,
wherein the specific voice is an uttered voice including a specific word.

3. The information processing apparatus according to claim 1,
wherein the specific voice is a specific biological sound.

4. The information processing apparatus according to claim 1,
wherein the processor is configured to:
generate a plurality of pieces of the image specification information based on a plurality of voices uttered intermittently during the imaging period of the series of radiation images; and
associate the plurality of pieces of image specification information with the series of radiation images.

5. The information processing apparatus according to claim 1,
wherein the processor is configured to display a time position of the radiation image specified by the image specification information in a video composed of the series of radiation images.

6. The information processing apparatus according to claim 1,
wherein the processor is configured to change the image specification information based on a change operation of a time position of the radiation image specified by the image specification information in a video composed of the series of radiation images.

7. The information processing apparatus according to claim 1,
wherein the processor is configured to:
acquire a series of optical images that are continuously captured in parallel with the capturing of the series of radiation images; and
play back each of a radiation video composed of the series of radiation images and an optical video composed of the series of optical images such that images at the same time point are displayed.

8. The information processing apparatus according to claim 1,
wherein the processor is configured to:
extract some radiation images from the series of radiation images with the radiation image specified by the image specification information as a starting point or an ending point; and
generate a data file including the extracted some radiation images.

9. The information processing apparatus according to claim 8,
wherein the processor is configured to:
acquire relevant information related to imaging content of the series of radiation images; and
generate a data file in which the relevant information and the some radiation images are associated with each other.

10. An information processing method executed by at least one processor included in an information processing apparatus, the information processing method comprising:
acquiring a series of radiation images captured by performing continuous irradiation with radiation;
generating, in a case in which a voice uttered during an imaging period of the series of radiation images is a specific voice, image specification information for specifying a radiation image selected from among the series of radiation images according to a timing of occurrence of the voice; and
associating the image specification information with the series of radiation images.

11. A non-transitory computer-readable storage medium storing a program for causing at least one processor included in an information processing apparatus to execute:
acquiring a series of radiation images captured by performing continuous irradiation with radiation;
generating, in a case in which a voice uttered during an imaging period of the series of radiation images is a specific voice, image specification information for specifying a radiation image selected from among the series of radiation images according to a timing of occurrence of the voice; and
associating the image specification information with the series of radiation images.
